(19) United States
(12) Patent Application Publication
(10) Pub. No.: US 2012/0156711 A1
(43) Pub. Date: Jun. 21, 2012

(54) System and method that allows the joined performance of optoelectric and respirometric sensors for instant and accurate ascertainment of biochemical oxygen demand (BOD) in liquid industrial wastes

(75) Inventors: Manuel Eduardo Young Anze, Valparaiso (CL); Cesar Raul Galindo Urra, Valparaiso (CL); Erika Mabel Valdes Carrion, Valparaiso (CL); Alejandro Mario Suarez Sotomayor, Valparaiso (CL); Fernando Antonio Albornoz Marquez, Valparaiso (CL); Cristian Andres Acevedo Gutierrez, Valparaiso (CL)

(73) Assignee: UNIVERSIDAD TECNICA FEDERICO SANTA MARIA, Valparaiso (CL)

(21) Appl. No.: 11/901,958

(22) Filed: Sep. 18, 2007

(51) Int. Cl. G01N 21/64; G01N 21/75; C12M 1/34

(52) U.S. Cl. 435/29; 435/288.7

(57) ABSTRACT

The present invention provides a system and method that combines the rapidity of the optoelectric signal and the simplicity of the respirometric cartridge wherein the greater accuracy of the respirometric biosensor is added to the rapidity of the optoelectric sensor, with the purpose of improving the accuracy of the measurements and the velocity of the integrated monitoring process and online control of the environmental variable of interest, removing the operative restrictions of work ranges.

### CROSS REFERENCE TO RELATED APPLICATIONS

None.

### STATEMENT REGARDING FEDERALLY SPONSORED RESEARCH OR DEVELOPMENT

This invention was not federally sponsored.

### FIELD OF THE INVENTION

The present invention relates to the environmental characterization of liquid wastes in general, such as activities of monitoring, control and also treatment. More specifically it relates to a self-calibration system and method that allows the joined performance of optoelectric and respirometric sensors for instant and accurate ascertainment of the Biochemical Oxygen Demand (BOD) in liquid industrial wastes (RILES).

### BACKGROUND OF THE INVENTION

The BOD (Biochemical Oxygen Demand) is an important index for monitoring organic pollution in liquids. It is an indirect measurement of the amount of organic material present in liquids in general and water in particular, which can be biologically degraded by microorganisms. Since the dissolved oxygen is completely consumed during the process of biochemical degradation of the organic material, its amount can be expressed, in an equivalent manner, in terms of the amount of oxygen required (respirometry). Thus, the BOD is able to identify in a quantitative manner the existing degradable load in waste waters or in a receptor body.

Depending on temperature conditions, other nutrients availability and absence of inhibitors, the whole degradation process normally takes approximately 20 days. As a partial analytical measure, but statistically representative of the organic load of a liquid waste, a conventional method has been used, denominated BOD₅, which consists of incubating the sample for 5 days at 20° C. Even though this is a normalized method, it is complicated and its major disadvantage is that the BOD quantification process takes 5 days, a lapse of time that does not allow for making timely and efficient decisions.

Due to the previously mentioned problem of response time in determining the BOD, it has been endeavored to perform almost instant online BOD determinations without waiting the 5 days of incubation for the conventional test to be performed.

For this reason, the prior art has utilized ultraviolet and fluorescence spectroscopic techniques that, when used together in a sequential manner, allow a determination of the BOD of a sample with high reproducibility and reliability in approximately 2 to 3 minutes.

The Chilean patent application, document CL 3256-2004 entitled “Determination of BOD in RILES using a UV-fluorescence method” describes optoelectric equipment for BOD determination in RILES (liquid industrial wastes) from industrial plants, in rivers, channels and lakes, wherein this equipment consists of two sensors, one for UV absorption and the other for fluorescence emission. Both work sequentially in the same sample and the combined data are processed by a nonlinear neuronal network. However, the error in determining the BOD using this technique is between 15 to 20% when compared to the BOD value determined by the normalized standard chemical method. While this is a considerable amount of error, the result is obtained in seconds.

The prior art has developed different types of biosensors based on respirometry and related methods that can determine the BOD of a sample in situ and in real time, and also to perform the measurement in a simple and rapid manner (in minutes). The biosensors consist of a combination of a transductor and a biological element, such as oxygen electrodes and microorganisms respectively.

The prior art on-line measuring systems consist basically of a unit (membrane or bioreactor) colonized by microorganisms (they can be specific to the liquid to be monitored), which is kept in a continuous flow due to a recirculation pump and fed with waste liquid by peristaltic pumps. Simultaneously, another pump saturates the liquid to be monitored (container fixed to the equipment or system) with oxygen, and a probe measures the dissolved oxygen in the liquid. This measurement can be performed with different configurations.

For instance, microorganisms immobilized in polyacrylamide gel and an oxygen electrode can be used (see “A Rapid method for Estimation of BOD by using Immobilized Microbial Cells” by Isao Karube et al. Biotechnology and Bioengineering Vol XIX, p. 1535-1547, 1977).

Also, the registry of the respirometric activity of the microorganisms in a reactor can be employed, which are extracted in a small amount (aliquot) from a chemostat.

STIP ISCO GmbH has a biosensor named biox 1010, which measures in an automatic manner the BOD and toxicity of water from different origins. The utilized method is respirometric, based on a microbial culture originated from the water to be monitored which is gradually deposited on insoluble supports inside the reactor (see EP 0369490), whose composition, concentration and activity are constant. It metabolizes the organic matter of the samples so that the oxygen consumed for its oxidation enables the determination of the BOD values and toxicity.

The apparatuses and methods previously described are deficient because they hold a limited amount of biomass which decreases the range of measurement of waters with low BOD. This is also the case with devices and methods that use membranes supported on the oxygen sensor. Additionally, devices and methods based on microorganisms supported on insoluble material must be generated in situ from the bacteria present in the liquid to be monitored. This makes it impossible to design the microbiological load of the reactor. Further, it requires a colonization period of many days that must be repeated every time the biofilm is deteriorated as a result of use or toxicity.

In the case of an apparatuses based on feeding from a chemostat, the operational requirements include a large number of feeding, draining, ventilation, recirculation, and cleaning pumps, which creates highly complex equipment that requires a long colonization time.

Another patent document is the application CL 84-2005 (PCT/EP2006/050235), entitled “Respirometry biosensor for rapid BOD determination in waters, which consists of a removable cartridge-type bioreactor (CTB), a system and method for rapid BOD determination.” It describes a cartridge that contains an adjustable number of capsules, which in turn hold immobilized adjustable masses of microorganisms, thereby permitting the design of a simple apparatus and use of a bioreactor with shorter colonization times. Reactors of this type can be cold stored allowing easy replacement of the operating reactor.

However, all the devices and methods previously described have shown to work with restrictions related to the range of operation and response time and not widely used in industrial applications. As of yet, a joined performance of optoelectric sensors and biosensors with control algorithms that allow its calibration has not been achieved. For that reason there, is not a dominant design for rapid BOD determination.

The advantage of the optoelectronic method is its immediateness of response, which can be within the range of 10 to 15 seconds; however, the obtained result is not extremely accurate. This value, though, is within an order of magnitude of the real BOD. The advantage of the respirometry method is that the response is direct, but it can take hours to obtain a BOD result.

### SUMMARY OF THE INVENTION

The present invention provides a system and method that combines the fast response time of the optoelectric signal and the simplicity of the respirometric cartridge wherein the greater accuracy of the respirometric biosensor is added to the speed of the optoelectric sensor, with the purpose of improving the accuracy of the measurements and the speed of the integrated monitoring process and on-line control of the environmental variable of interest and removing the operative restrictions of a fixed work range that cannot be set according to the BOD to be determined.

There has thus been outlined, rather broadly, the more important features of the invention in order that the detailed description thereof may be better understood, and in order that the present contribution to the art may be better appreciated. There are additional features of the invention that will be described hereinafter and which will form the subject matter of the claims appended hereto. The features listed herein and other features, aspects and advantages of the present invention will become better understood with reference to the following description and appended claims. The accompanying drawings, which are incorporated in and constitute part of this specification, illustrate embodiments of the invention and, together with the description, serve to explain the principles of the invention.

It should be understood that while the preferred embodiments of the invention are described in some detail herein, the present disclosure is made by way of example only and that variations and changes thereto are possible without departing from the subject matter coming within the scope of the following claims, and a reasonable equivalency thereof, which claims I regard as my invention.

### BRIEF DESCRIPTION OF THE FIGURE

FIG. 1 shows a general scheme of the system of the present invention.

### DETAILED DESCRIPTION OF THE INVENTION

In this manner, the present invention provides a system and method that integrates optoelectronic sensors and biosensors, wherein the first step combines the instant methodology with the optoelectric sensors that enables a user to set the boundaries or define the range of work requested to the biosensor. The boundaries or range of work will depend on the organic load of the sample, which at the same time will determine, as a second step, the volume of the sample entering the respirometry biosensor. The volume of the sample will also be adapted to the measurement range of the cartridge.

In fact, both the methodology and device work in parallel form, out of phase by a small period of time that is equal to the processing of the RIL (liquid industrial waste) sample. This sample is analyzed by the sensors, such as UV, fluorescence, or both, which are denominated optoelectronic sensors, such as the ones described in the patent application CL 3256-2004, wherein the sample is processed in a processing unit, according to the organic load of the analyzed sample.

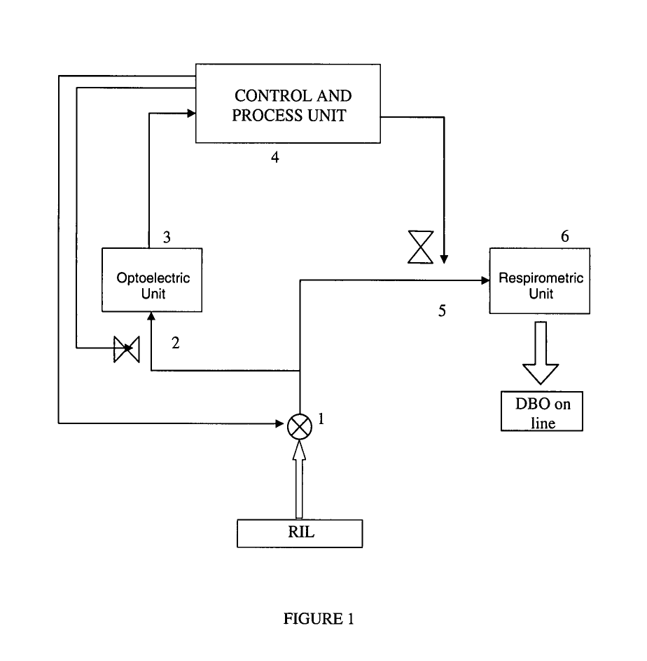

As shown in FIG. 1, a sample is taken from the RIL, through a pump (1) as a preferred form, which is controlled by a process and control unit (4), and the sample is distributed at the entrance of a valve (2) of an optoelectronic unit (3) of any type (UV, fluorescence, or combined). This optoelectronic unit (3) has an interface to convert the optic data to digital electronic signals or analogous signals, though preferably digital signals, which enter into the processing and control unit (4).

The received data are processed by the process and control unit (4) to determine with certainty the type of organic load of the sample and a computational program decides whether to apply a simple linear correlation, polynomial correlation, or neuronal networks, as the one disclosed in the patent application CL 3256-2004.

In this manner, a microprocessor defines, through specialized algorithms, a range of work for the respirometric unit (6), which is achieved by controlling the opening of the entrance valve (5) of the respirometric unit (6), thereby defining the entrance volume, such as in ml., to the respirometric unit (6).

The respirometric unit can be similar to the one defined in the application CL 84-2005. In this manner, the BOD that is determined will not exceed the upper limit (saturation) or lower limit (does not measure) of the measuring range of the removable and disposable cartridge type biosensor (BTC). The biomass, located inside of the disposable cartridge type biosensor, is encapsulated in a polymeric organic matrix suspended in a maintenance solution, where the maintenance solution is preferably calcium chloride.

In this manner it is achieved a fast and accurate measurement of BOD of the RILES analyzed by the present invention. The results can be obtained locally. In other words, the results can be read directly from the device. Alternatively, the results can be read remotely wherein the wireless means of transmitting the results is preferred. However, the transmission of the results can be accomplished through cables if wireless means are not possible.

What is claimed is:

1. A measuring system for rapid and accurate determination of BOD (biochemical oxygen demand) in water and/or RILES (liquid industrial wastes), wherein it comprises:
a pump with a respective valve, where the pump is able to take a sample from the water and/or RILES;
an optoelectric unit, where the optoelectric unit has an interface to convert optic data to electronic signals;
a process and control unit, where the process and control unit receives electronic signals and processes them through a microprocessor, where the process and control unit can then define the range of a working volume such that the BOD that is determined will not exceed the upper limit or lower limit of the measuring range of the measuring system; and
a respirometric unit, where the respirometric unit receives the working volume from the process and control unit.

2. The measuring system for rapid and accurate BOD determination according to claim 1, wherein the processing and control unit processes the RILES sample through the microprocessor to determine the organic load concentration of the sample in terms of a referential BOD.

3. The measuring system for rapid and accurate BOD determination according to claim 2, wherein the organic load concentration present in the sample in terms of the referential BOD is used by a computational program that decides whether to apply a simple linear correlation, a polynomial correlation, or neuronal networks.

4. The measuring system for rapid and accurate BOD determination according to claim 3, wherein the processing and control unit further comprises an entrance valve, where the processing and control unit controls the opening of the entrance valve to the respirometric unit, thereby determining the entrance volume of the sample used by the respirometric unit.

5. The measuring system for rapid and accurate BOD determination according to claim 1, wherein the optoelectronic unit comprises a UV sensor, fluorescence sensor, or both.

6. The measuring system for rapid and accurate BOD determination according to claim 1, wherein the respirometric unit comprises a removable and disposable cartridge-type biosensor (BTC).

7. The measuring system for rapid and accurate BOD determination according to claim 1, wherein the respirometric unit contains microorganisms immobilized in polyacrylamide gel and an oxygen electrode.

8. The measuring system for rapid and accurate BOD determination according to claim 1, wherein the respirometric unit comprises microorganisms in a reactor, which are extracted in a small amount from a chemostat.

9. The measuring system for rapid and accurate BOD determination according to claim 1, wherein the respirometric unit is based on a microbial culture originated from the water and/or RILES to be monitored, which is gradually deposited on insoluble supports inside a reactor.

10. A method for rapid and accurate determination of BOD (biochemical oxygen demand) in water and/or RILES, wherein it comprises the following steps:
(a) take a sample from the RILES using a pump;
(b) send the sample to an optoelectronic unit;
(c) convert the optic data to electronic signals;
(d) send the electronic signals to a process and control unit;
(e) process the electronic signals in a microprocessor that defines the range of a working volume, where the process and control unit defines the range of working volume such that the BOD that is determined will not exceed the upper limit or lower limit of the measuring range of the method; and
(f) send the range of working volume to a respirometric unit thereby obtaining a fast and accurate BOD measurement.

11. The method for rapid and accurate determination of BOD according to claim 10, wherein step (e), the step of processing the electronic signals in a microprocessor comprises determining the organic load concentration of the sample in terms of a referential BOD.

12. The method for rapid and accurate determination of BOD according to claim 11, wherein step (e), the step of processing the electronic signals further comprises the application of a simple linear correlation, polynomial correlation, or neuronal networks according to the concentration of organic load present in the sample in terms of the referential BOD.

13. The method for rapid and accurate determination of BOD according to claim 10, further comprising the step of:
(g) controlling the opening of an entrance valve to the respirometric unit and thereby allows the working volume of the sample to enter the respirometric unit.

14. The method for rapid and accurate determination of BOD according to claim 10, wherein the optoelectronic unit comprises a UV sensor, fluorescence sensor, or both.

15. The method for rapid and accurate determination of BOD according to claim 10, wherein the respirometric unit comprises a removable and disposable cartridge-type biosensor (BTC).

16. The method for rapid and accurate determination of BOD according to claim 10, wherein the respirometric unit comprises microorganisms immobilized in polyacrylamide gel and an oxygen electrode.

17. The method for rapid and accurate determination of BOD according to claim 10, wherein the respirometric unit comprises microorganisms in a reactor, which are extracted in a small amount from a chemostat.

18. The method for rapid and accurate determination of BOD according to claim 10, wherein the respirometric unit is based on a microbial culture originated from the water and/or RILES to be monitored and which is gradually deposited on insoluble supports inside the reactor.

19. A system for determining the biochemical oxygen demand in a liquid comprising:
a means for obtaining a sample of the fluid, an optoelectric unit, a process and control unit, a respirometric unit, and a means for displaying results to a user,
where the sample comprises an organic load,
where the optoelectric unit can collect optic data on the organic load of the sample, and where the optoelectric unit can convert the optic data to digital electronic signals,
where the process and control unit comprises a microprocessor, where the process and control unit can process the digital electronic signals from the optoelectric unit, where the process and control unit can then define a working volume for the respirometric unit,
where the respirometric unit uses a sample size defined by the working volume, where the respirometric unit determines the biochemical oxygen demand of the sample, and
where the means for displaying results to the user can display the resulting determination of the biochemical oxygen demand to the user.

20. The system of claim 19, wherein the processing and control unit processes the sample through the microprocessor to determine the organic load concentration of the sample in terms of a referential BOD,
where the organic load concentration present in the sample in terms of the referential BOD is used by a computational program that decides whether to apply a simple linear correlation, a polynomial correlation, or neuronal networks, and
where the processing and control unit further comprises an entrance valve, where the processing and control unit controls the opening of the entrance valve to the respirometric unit, thereby allowing the working volume of the sample to enter the respirometric unit.
